(19) United States
(12) Patent Application Publication
(10) Pub. No.: US 2023/0398266 A1
(43) Pub. Date: Dec. 14, 2023

(54) THERAPEUTIC AGENT FOR USE IN LOCAL TREATMENT OF INFLAMMATION ASSOCIATED WITH ATHEROSCLEROSIS AND/OR A THROMBOTIC STATE

(71) Applicant: UL1SSE INNOVATIVE SOLUTIONS S.R.L., MILANO (IT)

(72) Inventor: Alaide CHIEFFO, MILANO (IT)

(21) Appl. No.: 18/248,075

(22) PCT Filed: Oct. 5, 2021
(86) PCT No.: PCT/EP2021/077463

§ 371 Date: Apr. 6, 2023

(30) Foreign Application Priority Data
Oct. 6, 2020 (IT) 102020000023476

(51) Int. Cl. A61L 31/16; A61L 29/16; A61L 29/08; A61L 31/10; A61L 33/00

(52) U.S. Cl. CPC A61L 31/16; A61L 29/16; A61L 29/085; A61L 31/10; A61L 33/0005; A61L 2300/622; A61L 2300/422; A61L 2300/41; A61L 2300/256; A61L 2300/426

(57) ABSTRACT

A therapeutic agent which inhibits cytokines and/or inflammatory mediators for use in the treatment of a patient affected by inflammation associated with atherosclerosis and/or a thrombotic state, wherein the treatment includes the local administration of the therapeutic agent in a blood vessel by an intravascular medical device directly at the level of an atherosclerotic plaque, and/or a site of inflammation, and/or a site of a thrombotic phenomenon.

### CROSS-REFERENCE TO RELATED APPLICATIONS

This application is a 35 U.S.C. § 371 National Stage patent application of PCT/EP2021/077463 filed 5 Oct. 2021, which claims the benefit of Italian patent application 102020000023476 filed 6 Oct. 2020, the disclosures of which are incorporated herein by reference in their entirety.

### TECHNICAL FIELD

The present disclosure relates in general to a therapeutic agent for use in the local treatment of inflammation associated with atherosclerosis and/or a thrombotic state.

### BACKGROUND

A systemic and/or local inflammatory state caused by a viral or bacterial infection, by a systemic disorder or by causes that are not well known can trigger the development of local thrombotic phenomena affecting blood vessels.

In fact, it is known that a local inflammatory process can lead to endothelial dysfunction, hypercoagulability and consequently to a prothrombotic state involving arterial and/or venous vessels. For example, by means of this mechanism the inflammatory process can cause acute destabilization of the atherosclerotic plaque [Libby P. N Eng J Med 2013; 68:2004-13].

Atherosclerosis is a chronic inflammatory disease, characterized by a progressive accumulation of lipids within the arterial wall as a result of a local inflammatory process [Hansson G. N Engl J Med 2005; 352:1685-95]. The process that leads to atherosclerotic plaque formation has been described in numerous studies [Abrams J. N Engl J Med 2005; 352:2524-33].

Atherothrombosis, defined as atherosclerotic plaque rupture with superimposed thrombosis, has a pathophysiology characterized by the development over the years, at the level of the arterial vessels, of the atherosclerotic plaque. The atherosclerotic plaque may undergo a sudden destabilization, with consequent rupture/erosion and formation of an intravascular thrombus, leading to acute ischemia of the territory revascularized by the vessel occluded by thrombosis. Thus, atherothrombosis is the mechanism responsible for acute ischemia.

Acute myocardial ischemia manifests clinically with acute myocardial infarction, which remains one of the main causes of death globally. Besides being an important cause of mortality, acute myocardial infarction has a significant impact on the work capacity and quality of life of patients who survive the event. This aspect highlights the impact of atherothrombosis on public health.

However, the inflammatory process can be responsible for intravascular thrombosis even in the absence of an atherosclerotic plaque, for example in the case of inflammation resulting from a viral or bacterial infection. In particular, intravascular thrombosis of pulmonary arteries has been described as a possible pathophysiological substrate of the so-called “acute respiratory distress syndrome” that ensues in the case of infections such as for example that caused by SARS-CoV-2 (Severe Acute Respiratory Syndrome Coronavirus 2) [Lodigiani, Thromb Res 2020, Ciceri, Crit Care Resusc. 2020]. The presence of distal microthrombi in tributary vessels of ground glass areas in patients affected by COVID-19 (Coronavirus disease 2019) [Ciceri, Crit Care Resusc. 2020] further reinforces the hypothesis that a local inflammatory process may play an essential role in the development of this disease.

In addition to preventive strategies based on patient lifestyle changes and the control of cardiovascular risk factors with and without the use of drugs (such as aspirin and hypolipidemic agents), current therapeutic strategies for the treatment of atherothrombosis have focused on the treatment of the clinical manifestation of atherosclerotic disease (i.e., ischemia) at the time of its onset. This manifestation varies, depending on the vascular bed of interest, from myocardial infarction to cerebral stroke and limb ischemia. Current therapies are therefore aimed at eliminating the causes of flow obstruction (i.e., atherosclerotic plaque and thrombus) through reperfusion, both medical (systemic thrombolysis) and mechanical (primary angioplasty).

In order to prevent the evolution of the atherosclerotic pathology over time, with a consequent substantial clinical benefit for patients, a change in therapeutic strategy from treating the manifestation of atherosclerotic pathology (i.e., ischemia) to the pathophysiology of the process (i.e., atherothrombosis) would therefore be useful. Since inflammation is the underlying mechanism of atherothrombosis, it has been hypothesized that having inflammation as a target might influence the progression of atherosclerosis.

Anecdotal cases of patients treated with systemic administration of therapeutic agents having a strong anti-inflammatory action, including monoclonal antibodies directed against several cytokines, with reduction of atherosclerotic plaque, have been reported in the medical literature [Joshi A, Eur Heart J 2019, doi:10.1093/eurheartj/ehz349; Elnabawi Y A et al Cardiovasc Res. 2019; 115(4):721-728, doi:10.1093/cvr/cvz009].

Furthermore, the effects of systemic administration of canakinumab (an anti-interleukin-1 monoclonal antibody) on the risk of cardiovascular events such as death from heart disease, myocardial infarction, and cerebral stroke have been assessed in a randomized clinical trial that included patients with previous myocardial infarction and elevated inflammatory indices [Ridker P M, N Engl J Med 2017; 377:1119-31]. In this study, patients taking canakinumab had a lower incidence of cardiovascular events compared to placebo. However, a higher incidence of serious side effects such as fatal sepsis and thrombocythemia was observed in patients taking canakinumab compared to placebo.

Moreover, in patients with COVID-19 encouraging results have been shown in preliminary studies with systemic administration of anti-interleukin-1 (anakinra) or anti-interleukin-6 (tocilizumab) therapeutic agents; however, also in this case severe side effects related to systemic administration of the drugs were observed [Huet et al Lancet Rheumatol 2020; Guaraldi et al Lancet Rheumatol 2020].

### SUMMARY

In view of the above, the aim of the present disclosure is to provide a therapeutic agent for use in the treatment of inflammation associated with atherosclerosis and/or a thrombotic state which acts effectively on the pathophysiological process of atherothrombosis and/or thrombosis, limiting at the same time the side effects linked to the systemic administration of the therapeutic agent.

Within this aim, the disclosure provides a therapeutic agent for use in the treatment of inflammation associated with atherosclerosis and/or a thrombotic state which allows to prevent atherosclerotic pathology and/or thrombosis.

The present disclosure avoids or in any case reduces the need for therapeutic treatments directed against the clinical manifestation of atherosclerotic pathology (ischemia) and thus the need to carry out systemic thrombolysis and primary angioplasty procedures in patients.
the disclosure also provides a therapeutic agent for use in the treatment of inflammation associated with atherosclerosis and/or a thrombotic state, the administration of which to the patient relies on known medical devices commonly used in cardiovascular therapy.

This aim and these and other advantages which will become better apparent hereinafter are achieved by providing a therapeutic agent for use in the local treatment of an inflammation in a patient,
wherein the inflammation is associated with at least one condition selected from atherosclerosis and a thrombotic state, and
wherein the therapeutic agent inhibits one or more molecules selected from the group consisting of cytokines and inflammatory mediators and is administered locally in a blood vessel at the level of one or more of an atherosclerotic plaque, a site of inflammation and a site of a thrombotic phenomenon, by means of an intravascular medical device comprising a supporting structure and the therapeutic agent.

### DETAILED DESCRIPTION OF THE DISCLOSURE

Further characteristics and advantages of the disclosure will become better apparent from the following detailed description.

The present disclosure relates to the administration of therapeutic agents capable of inhibiting the action of interleukins and other inflammatory mediators at the local level, in arteries and/or veins which exhibit atherosclerotic and/or inflammatory phenomena and/or thrombi and/or microthrombi. For administration at the local level, local delivery by means of an intravascular device.

The medical literature mentioned previously clearly shows the role of inflammation as a mechanism underlying both atherothrombosis in the presence of atherosclerotic plaque and vascular thrombosis in the absence of atherosclerotic plaque (as a consequence, for example, of a viral and/or bacterial infection). Said literature, moreover, has provided evidence of positive effects of the administration of therapeutic agents which inhibit the action of interleukins or other molecules mediating inflammation in patients with atherosclerosis or COVID-19, highlighting however the presence of undesirable side effects of systemic treatment with said agents.

Without being bound to any theory in particular, on the basis of the above it is believed that therapeutic agents inhibiting one or more molecules selected from the group consisting of cytokines and inflammatory mediators, administered at the local level of blood vessels which exhibit phenomena of the atherosclerotic and/or thrombotic type by using an intravascular device, are capable of having an effective action on the pathophysiological process of atherothrombosis and/or thrombosis, at the same time limiting the undesirable side effects associated with their systemic administration.

According to the present disclosure, the therapeutic agent is administrated locally in a blood vessel (artery and/or vein) at the level of (i.e., at) one or more of an atherosclerotic plaque, a site of inflammation or a site of a thrombotic phenomenon (thrombi and/or microthrombi), by means of an intravascular medical device. In this manner, the therapeutic agent is administered directly on the (intracoronary or intra-arterial) atherosclerotic plaque, to the local inflammation site or to the site of the thrombotic phenomenon, inhibiting the ongoing inflammatory processes, in particular those inflammatory processes tied to the formation of atherosclerotic plaque and/or of the thrombotic phenomenon, as well as the inflammatory processes leading to destabilization, with consequent erosion and/or rupture, of the atherosclerotic plaque.

As mentioned, the therapeutic agents for use according to the present disclosure inhibit the action of one or more molecules selected from the group of cytokines and of inflammatory mediators. This inhibition can be the result of a direct interaction between the therapeutic agent and the cytokine or the inflammatory mediator (for example when the therapeutic agent is a monoclonal antibody targeting the cytokine or the mediator), by means of which the cytokine or the mediator is no longer capable of performing its biological action. As an alternative, the inhibition can also be the result of an interaction between the therapeutic agent and the specific cell receptor for the cytokine or the inflammatory mediator, as a result of which the receptor is no longer available for the binding to the cytokine or mediator molecule that is necessary for said molecule to be able to perform its biological action.

In one embodiment, the molecules selected from the group of cytokines and inflammatory mediators can be one or more of the following: interleukin 1 (IL-1), interleukin 6 (IL-6), interleukin 8 (IL-8), tumor necrosis factor alpha (TNF-α), C-X-C chemokine receptor type 4 (CXCR4), vascular cell adhesion molecule 1 (VCAM-1), C-C chemokine ligand 5 (CCL5), osteopontin (OPN), oxidized low-density lipoprotein receptor 1 (LOX1), lymphocyte-associated antigen 96 (LY96), integrin alpha M (CD11b), and heme oxygenase (HMOX).

The therapeutic agent for use according to the present disclosure can be a biological agent (so-called “biological drugs”), such as in particular monoclonal antibodies (for example humanized, fully human or chimeric) and fusion proteins (for example, fusions of a cell receptor or a subunit thereof with the Fc fragment of an immunoglobulin). More preferably, the therapeutic agent can be a monoclonal antibody.

As an alternative, the therapeutic agent for use according to the present disclosure can be an agent of the “traditional” type (so-called “synthetic drugs”), i.e., obtained by chemical synthesis starting from chemical and/or plant-derived compounds rather than biologically. For example, non-steroidal anti-inflammatory drugs (NSAIDs) and plant-derived anti-inflammatory drugs belong to this category of therapeutic agents. A plant-derived anti-inflammatory agent of particular interest is colchicine, the usefulness of which has been observed following systemic administration in the treatment of myocardial infarction [Tardif J C et al., N Engl J Med., 2019 Dec. 26; 381(26):2497-2505; Chiarito M et al., New Engl J Med 2020 Apr. 23; 382(17):1667-1668], but also in patients hospitalized with COVID-19 [Deftereos S et al., JAMA Netw Open 2020 Jun. 1; 3(6):e2013136].

By way of non-limiting example, the therapeutic agent for use in the present disclosure can be selected from the group consisting of adalimumab, anakinra, canakinumab, colchicine, etanercept, golimumab, infliximab, ixekizumab, leronlimab, rilonacept, sarilumab, secukinumab, siltuximab, tocilizumab, ulocuplumab, ustekinumab, and combinations thereof. Preferably, the agent can be selected from the group consisting of anakinra, canakinumab, colchicine, and tocilizumab.

In a preferred embodiment, the therapeutic agent for use according to the disclosure inhibits a cytokine selected from IL-1 and IL-6. In the context of said embodiment, the therapeutic agent is preferably selected from the group consisting of anakinra, canakinumab, sarilumab, siltuximab, and tocilizumab, and more preferably is selected from the group consisting of anakinra, canakinumab, and tocilizumab.

In another preferred embodiment, the therapeutic agent for use according to the disclosure is colchicine.

Naturally, in the context of the present disclosure the therapeutic agent can be formulated in a pharmaceutical composition which comprises, in addition to the therapeutic agent, one or more excipients or pharmaceutically acceptable carriers.

For local administration, the present disclosure is based on the use of an intravascular medical device which comprises a supporting structure and the therapeutic agent. The medical device can be introduced in the blood vessel (at the level of the atherosclerotic plaque, of the inflammation site or of the site of a thrombotic phenomenon) by percutaneous transcatheter route.

In one embodiment, the supporting structure of the intravascular medical device is selected from the group consisting of stents (for example, coronary stents and peripheral stents), catheters, vascular endoprostheses (for example, arteriovenous grafts and bypass grafts), and drug delivery balloons.

Catheters allow the direct administration of the therapeutic agent after cannulation of the blood vessel to be treated. The administration can be based on the direct injection of the therapeutic agent or can use technologies (such as microparticles or microspheres, but also ultrasound) which facilitate the absorption of the therapeutic agent at the level of the vascular wall.

Drug delivery balloons allow instead to transfer the therapeutic agent directly from the surface of the balloon to the vascular wall, by direct contact of the balloon with the vascular endothelium resulting from the inflation of said balloon. The release of the therapeutic agent is based on the presence of a carrier on the surface of the balloon, which allows the therapeutic agent to be absorbed in the vascular tissue. By way of example, in some drug delivery balloons currently used in order to reduce the risk of restenosis after coronary angioplasty (by release of an antiproliferative drug such as sirolimus or paclitaxel) the carrier is one of acetyl tributyl citrate (ATBC), nordihydroguaiaretic acid (NDGA), butyryl triehexyl citrate (BTHC), shellac, urea, phospholipid-based agents, and polysorbate.

Preferably, in the present disclosure the therapeutic agent is released in a controlled manner at the level of the atherosclerotic plaque, of the inflammation site or of the site of a thrombotic phenomenon, for example by using technologies such as microparticles, microspheres, elution, ultrasound or inflation at the local level.

In one preferred embodiment, the therapeutic agent is contained in a coating applied to the supporting structure of the medical device. The function of the coating is to act as carrier for the controlled release of the therapeutic agent or as reservoir for the therapeutic agent that is administered at the level of the atherosclerotic plaque, of the inflammation site or of the site of a thrombotic phenomenon. In this embodiment, the therapeutic agent is therefore released by elution (for example in the case of stents or catheters) or by inflation (in the case of drug delivery balloons).

Preferably, the coating is made of a polymeric material. Any polymeric material in which the therapeutic agent is substantially soluble is suitable for the purposes of the disclosure. Therefore, for the purposes of the present disclosure said coating of polymeric material can be of the hydrophilic or hydrophobic type and may furthermore be biodegradable or non-biodegradable (i.e., biostable).

Biodegradable polymeric materials that can be used in the present disclosure are for example those selected from the group consisting of poly-L-lactic acid, poly-DL-lactic acid, polycaprolactone, polyhydroxybutyrate, polyglycolic acid, polyidioxanone, polyhydroxyvalerate, polyorthoesters, polylactic-co-glycolic acid (PLGA) copolymers, polyanhydrides, and mixtures thereof.

Biostable polymeric materials that can be used in the present disclosure are for example those selected from the group consisting of polyurethanes, silicones, polyolefins, polyamides, polyvinyl chloride, polyvinyl alcohol, (meth)acrylic polymers and copolymers, polyacrylonitrile, polyethers, cellulose acetate, parylene, and mixtures thereof.

In another preferred embodiment, the therapeutic agent is encapsulated in microparticles or microspheres made of a polymeric material. For the purposes of the present disclosure, the microparticles or microspheres can be made of a biodegradable or biostable polymeric material. Examples of biodegradable or biostable polymeric materials with which the microparticles or microspheres can be provided are the same mentioned previously as suitable for the provision of a coating of polymeric material on the supporting structure.

In anther embodiment, the medical device can further comprise a device for emitting ultrasound. For example, so-called “ultrasound catheters” are known in the medical literature [Kaymaz C et al., Curr Vasc Pharmacol. 2018 Jan. 26; 16(2):179-189]. The acoustic waves of ultrasound increase the penetration capacity of the therapeutic agent and help to enhance the effectiveness of the treatment.

Furthermore, a preferred embodiment of the disclosure according to any one of the preceding embodiments is the one in which the inflammation is associated with atherosclerosis and the therapeutic agent is administered locally in a blood vessel at the level of an atherosclerotic plaque, so as to stabilize the atherosclerotic plaque and prevent its erosion or rupture.

Another preferred embodiment of the disclosure according to any one of the previously described embodiments is the one in which the inflammation is associated with a thrombotic state which is consequent to a viral or bacterial infection inducing at least one of local inflammatory phenomena and microthrombi in blood vessels (such as for example in the case of SARS-CoV-2 infection), and the therapeutic agent is administered locally in a blood vessel at the level of the site of local inflammatory phenomena and/or of the microthrombi.

Within the scope of any of the embodiments described above, the therapeutic agent for use according to the disclosure can be administered locally in combination with at least one additional therapeutic agent. In a preferred embodiment, the additional therapeutic agent co-administered locally is selected from the group consisting of antivirals, thrombolytics, anticoagulants, antithrombotics, antiaggregants, anti-inflammatory agents, and combinations thereof.

Finally, the present disclosure also relates to a method for the treatment of a patient affected by an inflammation associated with at least one condition selected from atherosclerosis and a thrombotic state, comprising the local administration of a therapeutic agent inhibiting one or more molecules selected from the group consisting of interleukins and inflammatory mediators in a blood vessel of the patient at the level of one or more of an atherosclerotic plaque, a site of inflammation and a site of a thrombotic phenomenon, by means of an intravascular medical device comprising a supporting structure and the therapeutic agent.

It should be understood that the features of all the embodiments described with reference to the therapeutic agent for use of the present disclosure are to be considered valid also regarding the above mentioned method of treatment, even if they are not expressly repeated.

The present disclosure fully achieves the intended aim and objects, since the local administration in a blood vessel of a therapeutic agent which inhibits one or more molecules selected from the group consisting of cytokines and inflammatory mediators at the level of a blood vessel, at the level of one or more of an atherosclerotic plaque, a site of inflammation and a site of a thrombotic phenomenon, allows to inhibit the inflammatory processes underlying the formation of atherosclerotic plaque and/or thrombi, as well as the destabilization of atherosclerotic plaque.

Furthermore, by using the local administration of the therapeutic agent, the present disclosure allows to avoid or at least reduce the undesirable side effects observed in the case of systemic administration of therapeutic agents inhibiting cytokines or inflammatory mediators.

Moreover, by acting directly on the mechanism underlying atherosclerosis and thrombosis before an ischemia occurs in the patient, the present disclosure provides a therapy that is effective in preventing atherosclerotic pathology and/or thrombosis.

Consequently, the present disclosure allows to avoid or in any case reduce the need for patients to undergo systemic thrombolysis and primary angioplasty procedures.

Finally, by making use of medical devices already used in the cardiovascular field (such as, for example, stents, catheters and drug delivery balloons) for the local administration of the therapeutic agent, the present disclosure provides a therapy for the treatment of inflammation associated with atherosclerosis and/or a thrombotic state that is readily applicable, with the use of widely tested surgical techniques (using, for example, a percutaneous transcatheter) to introduce the medical device in the blood vessel of the patient.

The therapeutic agent for use and the method of treatment according to the present disclosure are susceptible of numerous modifications and variations, all of which are within the scope of the inventive concept. All the details may furthermore be replaced with other technically equivalent elements.

1-10. (canceled)

11. A method for local treatment of a patient affected by an inflammation associated with at least one condition selected from atherosclerosis and a thrombotic state, the method including the following step:
locally administering a therapeutic agent inhibiting one or more molecules is selected from the group consisting of cytokines and inflammatory mediators in a blood vessel at the level of one or more of an atherosclerotic plaque, a site of inflammation and a site of a thrombotic phenomenon, by an intravascular medical device comprising a supporting structure and the therapeutic agent.

12. The method according to claim 11, wherein the intravascular medical device comprises a supporting structure selected from the group consisting of stents, catheters, endoprostheses, and drug delivery balloons.

13. The method according to claim 11, wherein the therapeutic agent is contained in a coating applied to the supporting structure.

14. The method according to claim 13, wherein the coating is made of a biodegradable or non-biodegradable polymeric material.

15. The method according to claim 11, wherein the therapeutic agent is encapsulated in microspheres made of a polymeric material.

16. The method according to claim 11, wherein the therapeutic agent inhibits one or more molecules selected from the group consisting of interleukin 1 (IL-1), interleukin 6 (IL-6), interleukin 8 (IL-8), tumor necrosis factor alpha (TNF-α), C-X-C chemokine receptor type 4 (CXCR4), vascular cell adhesion molecule 1 (VCAM-1), C-C chemokine ligand 5 (CCL5), osteopontin (OPN), oxidized low-density lipoprotein receptor 1 (LOX1), lymphocyte-associated antigen 96 (LY96), integrin alpha M (CD11b), and heme oxygenase (HMOX).

17. The method according to claim 11, wherein the therapeutic agent is selected from the group consisting of adalimumab, anakinra, canakinumab, colchicine, etanercept, golimumab, infliximab, ixekizumab, leronlimab, rilonacept, sarilumab, secukinumab, siltuximab, tocilizumab, ulocuplumab, ustekinumab, and combinations thereof.

18. The method according to claim 11, wherein the therapeutic agent is administered locally in combination with a further therapeutic agent selected from the group consisting of antivirals, thrombolytics, anticoagulants, antithrombotics, antiaggregants, anti-inflammatory agents and combinations thereof.

19. The method according to claim 11, wherein the inflammation is associated with a thrombotic state which is consequent to a viral or bacterial infection inducing at least one of local inflammatory phenomena and microthrombi in blood vessels and the therapeutic agent is administered locally in a blood vessel at the level of one or more of the site of the local inflammatory phenomena and of the microthrombi.

20. The method according to claim 11, wherein the inflammation is associated with atherosclerosis and the therapeutic agent is administered locally in a blood vessel at the level of an atherosclerotic plaque, stabilizing the atherosclerotic plaque and preventing erosion or rupture of the atherosclerosis plaque.
